(19) United States
(12) Patent Application Publication
(10) Pub. No.: US 2009/0054634 A1
(43) Pub. Date: Feb. 26, 2009

(54) Process for the preparation of clarithromycin

(76) Inventors: Vinod Kumar Kansal, Haryana (IN); Dhirenkumar N. Mistry, Gujarat (IN); Mitesh Gandhi, Gujarat (IN); Rakesh Ravjibhai Patel, Greater Noida (IN)

Correspondence Address: KENYON & KENYON LLP, ONE BROADWAY, NEW YORK, NY, 10004, US

(21) Appl. No.: 12/228,297

(22) Filed: Aug. 11, 2008

Related U.S. Application Data
(60) Provisional application No. 60/935,380, filed on Aug. 9, 2007.
(60) Provisional application No. 61/019,472, filed on Jan. 7, 2008.

(51) Int. Cl. C07H 17/08

(52) U.S. Cl. 536/7.2

(57) ABSTRACT

The present invention includes a process involving a one-pot reaction for preparing erythromycin 9-oxime salt comprising: (a) reacting erythromycin thiocyanate with an ammonium source to obtain erythromycin free base; (b) oximating the C-9 carbonyl of the erythromycin free base by reacting the erythromycin free base with triethylamine and hydroxyl amine hydrochloride to form erythromycin oxime; and (c) reacting the erythromycin oxime obtained in step (b) with an ammonium source to obtain the erythromycin 9-oxime salt. The present invention is also drawn to a one-pot reaction for preparing clarithromycin starting with the one-pot reaction for preparing erythromycin 9-oxime salt, further comprising after step (c): (d) silylating the hydroxy groups at the oxime group, and the 2′ and 4″ positions of the erythromycin 9-oxime salt to obtain a silylated derivative; (e) methylating the hydroxy group at the 6 position of the silylated derivative using at least one methylating agent in the presence of at least one inorganic base to obtain SMOP, wherein SMOP is 6-O-methyl-2′,4″-bis(trimethylsilyl)-erythromycin A 9-O-(2-methoxyprop-2-yl)oxime; and (f) converting the SMOP into clarithromycin using at least one deoximating agent in the presence of aqueous ethanol.

### CROSS REFERENCE TO RELATED APPLICATIONS

The present application claims the benefits of U.S. Provisional Application Nos. 60/935,380 filed Aug. 9, 2007 and 61/019,472 filed Jan. 7, 2008, the disclosures of both provisional applications are incorporated by reference.

### FIELD OF INVENTION

The present method relates to an improved method for the preparation of erythromycin 9-oxime salt and an improved method for the preparation of clarithromycin.

### BACKGROUND OF INVENTION

Clarithromycin (CLM) is a semi-synthetic macrolide antibiotic related to erythromycin A. It exhibits excellent anti-bacterial activity against gram-positive bacteria, some gram-negative bacteria, anaerobic bacteria, mycoplasma, and Chlamydia. It is stable under acidic conditions and is efficacious when administered orally. Clarithromycin is a useful therapy for infections of the upper respiratory tract in children and adults.

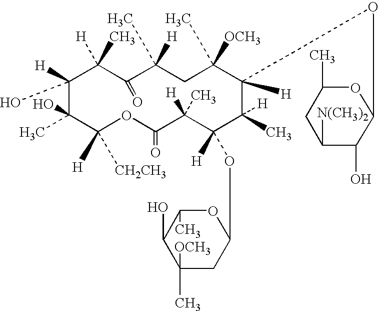

Various methods for the preparation of clarithromycin have been suggested. One of the most effective methods includes the following steps: protecting the 9-oxo group with a substituted oxime group, protecting the hydroxyl groups in positions 2′ and 4″, methylating the hydroxyl group in position 6 to give a protected silylated clarithromycin oxime, and removing the protecting groups at the 2′, 4″ and 9 positions.

U.S. Pat. No. 6,617,436 describes a process for the preparation of clarithromycin according to the following scheme:

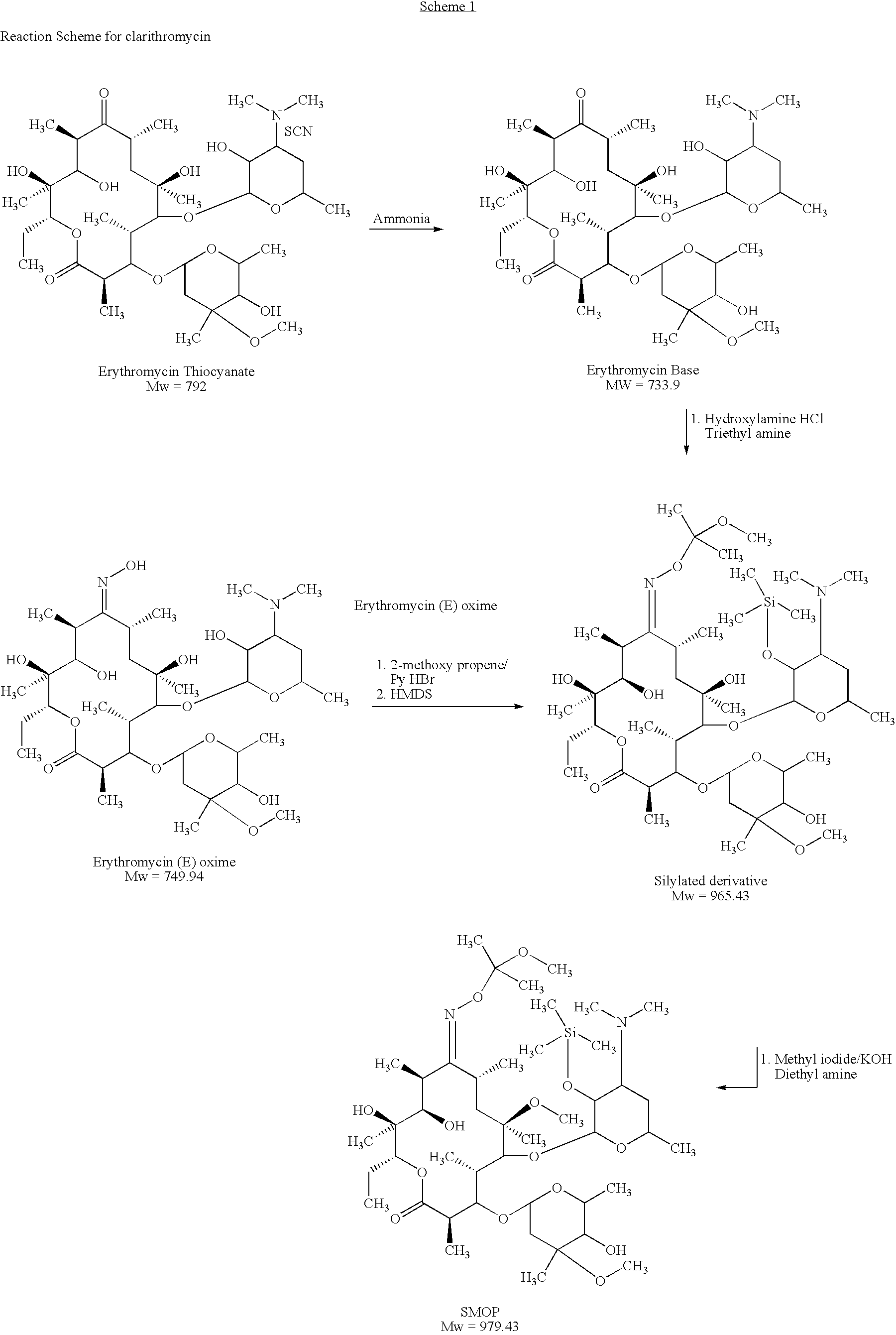

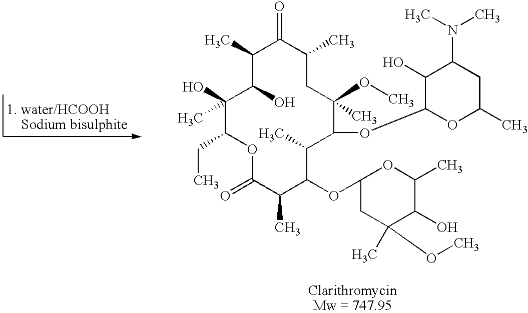

Scheme 1 describes the synthesis of clarithromycin from erythromycin thiocyanate. The process comprises the conversion of erythromycin thiocyanate to erythromycin base (A) in dichloromethane with ammonia and the isolation of pure erythromycin base. The isolated base is reacted with hydroxyl amine hydrochloride and triethyl amine in an alcoholic solvent to yield erythromycin oxime hydrochloride which was isolated after filtration; and the oxime free base has been prepared in water and acetone after adjusting the pH to 12 with ammonia and the separated solid is filtered and dried to give the erythromycin oxime base. The dried erythromycin base was converted to a silyl protected oxime derivative in dichloromethane in the presence of 2-methoxy propene and pyridine hydrobromide. The isolated silyl derivative is converted to SMOP in a biphasic system in the presence of methyl iodide and KOH to give the SMOP which is subsequently deprotected with deoximinated agent in aqueous ethanol, in the presence of formic acid to yield crude clarithromycin. Purification of the crude clarithromycin with ethanol resulted in the pure clarithromycin.

During the operation of isolation and drying of the erythromycin base from erythromycin thiocyanate, while removing the undesired impurities (Erythromycin—C, E, F, etc), the required erythromycin—A is also lost in the mother liquor. Similarly, during the conversion of erythromycin 9-A oxime hydrochloride to erythromycin 9-A-oxime base in water miscible organic solvents in the presence of ammonia, the required oxime is lost in the mother liquor, and hence the yield is reduced.

Many processes for the preparation of clarithromycin are disclosed in the prior art, for example WO 2006/064299 and WO 2006/100691. Neither of these methods, however, led to high yields.

### SUMMARY OF INVENTION

The present invention provides a one-pot reaction for the preparation of erythromycin 9-oxime salt.

The present invention also provides a one-pot reaction for the preparation of clarithromycin.

### DETAILED DESCRIPTION OF THE INVENTION

As used herein, the term “one pot reaction” refers to a reaction which includes two or more sequential reactions without the isolation of the intermediates, for example by filtration.

As used herein, the term “aprotic solvent” refers to an organic solvent that does not exchange protons with a substance dissolved in it.

The present invention provides a one-pot reaction for converting erythromycin thiocyanate to clarithromycin, which relates with increased product yield, and decreased cost.

In one embodiment, the present invention is drawn to a one-pot reaction for preparing erythromycin 9-oxime salt comprising:

(a) reacting erythromycin thiocyanate with an ammonium source to obtain erythromycin free base; and

(b) oximating the C-9 carbonyl of the erythromycin free base by reacting the erythromycin free base with triethylamine and hydroxyl amine hydrochloride to form the erythromycin 9-oxime salt.

Preferably, the erythromycin oxime salt is erythromycin oxime hydrochloride.

Preferably, the ammonium source is an aqueous ammonia solution. More preferably, the ammonia source is an about 25% (v/v) liquid ammonia solution. The reaction of step (a) can be carried out in the presence of at least one organic solvent, such as dichloromethane. Preferably, the organic layer of step (a) is removed from the aqueous layer, and further distilled under reduced pressure.

The reaction of step (b) can be carried out in the presence of at least one organic solvent, such as C₁-C₄ alcohols, preferably, methanol, isopropanol and ethanol, most preferably methanol. Preferably, the reaction mixture of step (b) is heated to about 60° C. to about 75° C., most preferably to about reflux, for about 12 hours to about 30 hours, most preferably for about 20 hours to about 24 hours. The mixture resulting from the reaction in step (b) can be further washed with cold methanol, wherein the cold methanol is at a temperature of about 15° C. or less.

In another embodiment, the present invention comprises a one-pot reaction for preparing clarithromycin comprising:

(a) reacting erythromycin thiocyanate with an ammonium source to obtain erythromycin free base;

(b) oximating the C-9 carbonyl of the erythromycin free base by reacting the erythromycin free base with triethylamine and hydroxyl amine hydrochloride to form an erythromycin 9-oxime salt;

(c) further reacting the erythromycin oxime with an ammonium source;

(d) silylating the hydroxy groups at the oxime group, and the 2′ and 4″ positions of the erythromycin 9-oxime salt using hexamethyl disilazene (HMDS) in the presence of methoxy propene and pyridine hydrobromide to obtain the silylated derivative;

(e) methylating the hydroxy group at the 6 position of the silylated derivative using a methylating agent in the presence of at least one inorganic base to obtain SMOP; and

(f) converting the SMOP into clarithromycin using a deoximating agent in the presence of an aqueous ethanol.

Preferably, the yield of the obtained clarithromycin is above 54%.

As used herein, “SMOP” stands for 6-O-methyl-2′,4″-bis(trimethylsilyl)-erythromycin A 9-O-(2-methoxyprop-2-yl)oxime.

Preferably, the erythromycin oxime salt is erythromycin oxime hydrochloride.

Preferably, the ammonium source is an about 25% (v/v) liquid ammonia solution. The reaction of step (a) can be carried out in the presence of at least one organic solvent, such as dichloromethane. Preferably, the organic layer of step (a) is removed from the aqueous layer, and further distilled under reduced pressure.

The reaction of step (b) can be carried out in the presence of at least one organic solvent, such as methanol. Preferably, the reaction mixture of step (b) is heated to about 60° C. to about 75° C., most preferably, to about reflux, for about 12 hours to about 30 hours, more preferably to about 20 hours to about 24 hours. The mixture resulting from the reaction in step (b) can be further washed with cold methanol, wherein the cold methanol is at a temperature of about 15° C. or less.

The reaction of step (c) can be carried out in the presence of at least one organic solvent, such as dichloromethane. Preferably the ammonia solution of step (c) is added in a dropwise manner.

Preferably, the reaction mixture of step (d) is maintained at a temperature of about 10° C. to about 20° C.

Step (e) can be carried out in the presence of at least one organic solvent. The at least one organic solvent of step (e) can include methyl tert-butyl ether, with or without another aprotic solvent. Most preferably, the another aprotic solvent is dimethylsulfoxide. The at least one inorganic base can be a base selected from a group consisting of potassium hydroxide, sodium hydroxide, potassium hydride, sodium hydride, potassium tert-butoxide, and sodium tert-butoxide. Most preferably, the at least one inorganic base is potassium hydroxide. The methylating agent is preferably an agent such as methyl iodide, methyl bromide, dimethylsulfate, methyl p-toluenesulfonate, methyl methanesulfonate, and dimethyl sulfate. The methylating agent is most preferably methyl iodide. In one specific embodiment, the reaction mixture in step (e) is maintained at a temperature of about 10° C. to about 20° C.

Suitable deoximating agents for step (f) include inorganic sulfur oxide compounds such as sodium hydrogen sulfite, sodium pyrosulfate, sodium thiosulfate, sodium sulfite, sodium hydrosulfite, sodium metabisulfite, sodium dithionate, potassium hydrogen sulfite, potassium thiosulfate, and potassium metabisulfite. Most preferably, the deoximating agent is sodium metabisulphite. The amount of deoximating agent can be about 1 to about 10 molar equivalents, preferably about 4 to about 7 molar equivalents, relative to the protected silylated clarithromycin oxime.

In one specific embodiment, step (f) includes reacting SMOP with an acid, such as formic acid, and a deoximating agent in the presence of aqueous ethanol at an ethanol/water ratio of about 1:1 to about 0.1:1 (v/v), heating the mixture to about 40° C. to about 85° C., more preferably to about 50° C. to about 65° C., cooling the reaction mixture and adding sodium hydroxide. The acid such as formic acid is preferably added until the pH of the reaction mixture reaches about 3.5 to about 4.5. After the acid addition, sodium hydroxide can be added until the pH of the reaction mixture reaches about 9 to about 12, more preferably about 10 to about 11, and most preferably about 10.2 to about 10.5.

Examples of the yield in percentage that can be achieved in the processes of the present invention are given below in Table 1:

TABLE 1

| | | | % (product |
|---|---|---|---|
| | | | weight/reagent |
| Sr. no | Name of Compound | % Yield | weight) |
| 1 | Erythromycin Thiocyanate | 92 | 1.21 |
| | to silyl ester | | |
| 2 | Silyl Ester to S-MOP | 94 | 0.95 |
| | Oxime | | |
| 3 | S-MOP Oxime to | 63 | 0.48 |
| | clarithromycin | | |
| | Overall yield | 54 | 0.55 |

In the processes of the present invention, the yield of the silyl ester based on erythromycin thiocyanate as the starting compound can be 92% or higher.

In the processes of the present invention, the yield of the SMOP based on the conversion of the silyl ester to SMOP oxime can be 94% or higher.

In the processes of the present invention, the yield of clarithromycin based on the conversion of SMOP oxime to clarithromycin can be 63% or higher.

In the processes of the present invention, the overall yield of clarithromycin based on erythromycin thiocyanate as the starting compound can be 54% or higher.

Examples of the ratio of the E/Z isomers formed in various steps in the processes of the present invention are given in Table 2:

TABLE 2
Percentage of E and Z Isomer in Clarithromycin Intermediates
in the Instant Invention.

| Sr. no | Name of Compound | % E | % Z |
|---|---|---|---|
| 1 | Erythromycin Oxime HCl | 89-96 | 3-8 |
| 2 | Silyl derivative | 72-78 | 2-8 |
| 3 | S-MOP | 67-75 | 2-8 |

### EXAMPLES

Having thus described the invention with reference to particular preferred embodiments and illustrative examples, those in the art may appreciate modifications to the invention as described and illustrated that do not depart from the spirit and scope of the invention as disclosed in the specification. The Examples are set forth below for demonstration purposes in order to aid in understanding the invention.

### Example 1

### In Situ Preparation of Clarithromycin from Erythromycin Thiocyanate

1.0 Kg Erythromycin thiocyanate was suspended in 5.6 L dichloromethane, and 1.33 L 25% liquid ammonia solution was added at a temperature of 25-35° C. The mixture was stirred so that the solid would completely dissolve. The lower dichloromethane layer was removed from the aqueous layer. The aqueous layer was extracted with 1.4 L dichloromethane. Both dichloromethane layers were combined and washed with process water. Dichloromethane was distilled out from the solution and traces of dichloromethane were removed by applying reduced pressure. 0.670 L methanol was added and then the methanol was distilled out to remove traces of dichloromethane and water. The viscous liquid was cooled to 25-35° C. 3.0 L Methanol, 0.3176 kg triethyl amine, 0.4387 kg hydroxyl amine hydrochloride were charged and the mass was heated to reflux. The mass was refluxed for 20-24 hrs. After completion of the reaction, the mass was cooled to 0-5° C. The product was cooled and washed with 0.1 L chilled methanol. The wet cake was unloaded and suspended in 8.47 L dichloromethane. 0.215 L 20-25% Liq ammonia was added slowly at 25-35° C. and stirred for 30 minutes. The dichloromethane layer was removed. The dichloromethane layer was washed with process water. 2.42 L Dichloromethane was distilled out from the mass to remove moisture. The mass was cooled down to 7-10° C. 0.331 Kg 2-Methoxy propene and 0.294 kg pyridine hydrobromide were added. The mass was stirred to 12-17° C. for 120 minutes, and then 0.371 kg hexamethyl disilazene was charged and the mass was stirred for 60 min. 2.42 L 8% Sodium bicarbonate solution was charged into the reaction mixture, which was stirred for 30 min at 27-33° C. The dichloromethane layer was separated out and washed with 2.42 L water. Dichloromethane was distilled out completely and then traces of dichloromethane were removed by adding 1.0 L ethyl benzene and recovered under reduced pressure. The residue was cooled to room temperature and 14.5 L methyl tert-butyl ether and 12.10 L dimethyl sulphoxide were added and the mass was cooled to 14-15° C. Under vigorous stirring, 1.85 kg methyl iodide and 1.21 kg powder potassium hydroxide were added. The mixture was stirred at 12-17° C. for 35-40 min. After completion of the reaction, 0.726 L dimethyl amine solution and 2.2 L process water were added. The mass was stirred for 30 minutes and the bottom aqueous layer was removed. The aqueous layer was extracted with 2.4 L methyl tert-butyl ether (MTBE). Both methyl tert-butyl ether layers were combined and washed with brine solution and water. Methyl ter-butyl ether was distilled out and to it were added 1.2 liters of water. Water and MTBE were distilled out to remove the traces completely. This residue was suspended in oxime in 3.3 liters ethyl alcohol, to which were added 3.0 liters of water at a temperature of 25° C.-35° C. to obtain a reaction mixture. To the reaction mixture, were added 1.27 kg of sodium metabisulphite and 0.173 kg of formic acid to adjust pH 3.8 to 4.1. The temperature of the mass was raised to 60° C. and stirred at 57-63° C. for 5 hrs and then cooled to a temperature of 50-55° C. 1.27 Kg Sodium metabisulphite was added. The mass was heated to 60° C. The mass was stirred and the temperature of the reaction was maintained at 57-63° C. for 5.0 hours. The mass was cooled to 30-35° C. and 50% NaOH solution to adjust pH 10.5 to 11.0, were slowly added, after cooling the mass down to the temperature of 30° C.-35° C. for 30 min. The resulting slurry was filtered and the cake was washed with 0.25 liters of ethanol and water in a ratio of 0.50:0.60 respectively. The resultant wet solid was stirred with 80.0 liters of water at 30-40° C. The crude clarithromycin was dried until the moisture content was less than 2%. Dry Weight: 0.68-0.65 kg.

Purification of Crude Clarithromycin 14 Liters of ethanol were charged with 1.0 kg of crude clarithromycin in a reactor. 0.1 Kg carbon and 0.1 kg hyflow were added after heating the mass to reflux. The resulting mass were filtered and washed with 1.0 liter of hot ethanol. The clear filtrate was collected and cooled up to 0-5° C., which was stirred at 0.5° C. for 2 hrs. The product was filtered and dried at 70-75° C. until the LOD was less than 2%.

### Example 2

### Synthesis of Erythromycin Oxime Base

To 1.0 kg erythromycin base, 2.5 L Methanol was added. To this solution 0.34 kg triethylamine, 0.47 kg hydroxyl amine hydrochloride were charged. The mass was heated to reflux temperature and refluxed for 20-24 hrs. After completion of the reaction, recover methanol 0.3 volume the reaction mass was cooled to 0-5° C. The product was filtered & washed with 0.1 L chilled methanol to obtain a wet cake. The wet cake was unloaded and suspended in 2.25 L Methanol. Slowly 0.65 L 20-25% Liq ammonia was added at 10-12° C. and stirred for 30 minutes; 0.65 L water was added to start precipitation, and 3.0 L water were added again in 60 minutes. The slurry was stirred for 30 minutes the slurry was filtered, and the slurry was washed with 2.0 L water. The wet cake was unloaded and dried at a temperature of 60-65° C. Yield=0.8 kg (78.4%).

### Synthesis of Silyl Ester.

4.0 L dichloromethane were added to 0.8 kg erythromycin oxime base, and the dichloromethane was distilled out from the mass for removing moisture. The resulting product was cooled down to 7-10° C. 0.232 Kg 2-methoxy propene and 0.208 kg pyridine hydrobromide were added. The mass was stirred to 12-17° C. for 120 minutes, and then 0.256 kg hexamethyl disilazene was charged and stirred for 60 min. 1.6 L 8% Sodium bicarbonate solution was charged into the reaction mixture and stirred for 30 min at 27-33° C. The dichloromethane layer was separated out, and then the dichloromethane layer was washed with 1.6 L water. The dichloromethane was distilled out in such a way that the distillation of dichloromethane and addition of 8 L water remained same. The mass was slowly heated up to 70-80° C. and vacuum was applied to remove traces of dichloromethane. The slurry was cooled to 15-20° C. and stirred for 2-3 hrs. The product was filtered and the wet product was dried at a temperature of 50-60° C. until the LOD was less than 0.5%. Dry weight=1.0 kg, 96.9%.

SMOP Oxime from Silyl Ester.

12 L Methyl tertiary butyl ether was charged, 1.00 kg of the silyl ester derivative was added at ambient temperature, and then the solution was cooled to 12-15° C. 10 L Dimethyl sulphoxide was added and the mass was cooled to 14-15° C. Under vigorous stirring 0.153 kg methyl iodide and 0.100 kg powder potassium hydroxide were added. The mixture was stirred at a temperature of 12-17° C. for 35-40 min. After completion of the reaction, 0.60 L dimethyl amine solution and 2 L process water were added. The mixture was stirred for 30 minutes and the bottom aqueous layer was removed. The aqueous layer was extracted with 2.0 L methyl tert-butyl ether, the methyl tert-butyl ether was combined and washed with brine solution and water. The methyl tert-butyl ether was distilled out and 8.0 L hot water was added to remove traces of the methyl tert-butyl ether. The slurry was cooled down to 20-25° C., filtered and washed with 1 L water. The wet product was dried at 50-55° C. until the moisture content was less than 2%. Dry weight=0.96 kg, 94.6%.

### Example 3

### Preparation of Clarithromycin

0.96 Kg SMOP oxime was suspended in 2.88 L ethyl alcohol. 2.88 L Water was added at 25-35° C. Then 0.559 kg sodium metabisulphite was added and 0.135 kg formic acid was added to adjust the pH to 3.8-4.1. The temperature of the mass was raised to 60° C. The mass was stirred at 57-63° C. for 5.0 hours, and then cooled to a temperature of 50-55° C. 0.559 Kg Sodium metabisulphite was added. The mass was heated to 60° C. The mass was stirred and the temperature of the reaction was maintained at 57-63° C. for 5.0 hours. The mass was cooled to 30-35° C. and a 50% NaOH solution was slowly added to adjust the pH to 10.5-11.0. The mass was stirred at 30-35° C. for 30 min. The slurry was filtered and washed with 0.25 L ethanol+water (0.50+0.60). The wet solid was stirred with 8.0 L water at 30-40° C. The product was dried until the moisture content was less than 2%. Dry weight=0.48 kg.

### Example 4

### Purification of Clarithromycin

6.8 L Ethanol and 0.48 kg crude clarithromycin were charged in a reactor. The mass was heated to reflux. 0.1 Kg Carbon and 0.1 kg hyflow were added. The carbon cake was filtered and washed with 0.48 L hot ethanol. The filtrate was collected and cooled up to 0-5° C. The filtrate was then stirred at 0-5° C. for 2 hrs. The product was filtered and dried at 70-75° C. until LOD was less than 2%. Yield=0.4 kg (54.56% from SMOP).

### Example 5

### Preparation of Silylated Derivative

1.0 Kg Erythromycin thiocyanate was suspended in 5.6 L dichloromethane. 1.33 L of 25% Liquid ammonia solution was added at a temperature of 25-35° C. The mixture was stirred, so that the solid would completely dissolve. The lower dichloromethane layer was separated from the aqueous layer. The aqueous layer was then extracted with 1.4 L dichloromethane. Both dichloromethane layers were combined and washed with process water. Dichloromethane was distilled out from the solution and traces of dichloromethane were removed by applying reduced pressure. 0.670 L Methanol was added. The methanol was distilled out to remove traces of dichloromethane and water. The viscous liquid was cooled to 25-35° C. 3.0 L Methanol, 0.3176 kg triethylamine, 0.4387 kg hydroxyl amine hydrochloride were charged. The mass was heated to reflux temperature and refluxed for 20-24 hrs. After completion of the reaction, the reaction mass was cooled to 0-5° C. The product was filtered & washed with 0.1 L chilled methanol to obtain a wet cake. The wet cake was unloaded and suspended in 8.47 L dichloromethane. Slowly 0.215 L 20-25% Liq ammonia was added at 25-35° C. and stirred for 30 minutes. The dichloromethane layer was separated out and then washed with process water. 2.42 L dichloromethane was distilled out from the mass for removing moisture. The resulting product was cooled down to 7-10° C. 0.331 Kg 2-methoxy propene and 0.294 kg pyridine hydrobromide were added. The mass was stirred to 12-17° C. for 120 minutes, and then 0.371 kg hexamethyl disilazene was charged and stirred for 60 min. 2.42 L 8% Sodium bicarbonate solution was charged into the reaction mixture and stirred for 30 min at 27-33° C. The dichloromethane layer was separated out, and then the dichloromethane layer was washed with 2.42 L water. The dichloromethane was distilled out in such a way that the distillation of dichloromethane and addition of 10 L water remained same. The mass was slowly heated up to 70-80° C. and vacuum was applied to remove traces of dichloromethane. The slurry was cooled to 15-20° C. and stirred for 2-3 hrs. The product was filtered and the wet product was dried at a temperature of 50-60° C. until the LOD was less than 0.5%. Dry weight=1.21 kg.

### Example 6

### Preparation of S-MOP

12 L Methyl tertiary butyl ether was charged, 1.00 kg of the silyl ester derivative was added at ambient temperature, and then the solution was cooled to 12-15° C. 10 L Dimethyl sulphoxide was added and the mass was cooled to 14-15° C. Under vigorous stirring 0.153 kg methyl iodide and 0.100 kg powder potassium hydroxide were added. The mixture was stirred at a temperature of 12-17° C. for 35-40 min. After completion of the reaction, 0.60 L dimethyl amine solution and 2 L process water were added. The mixture was stirred for 30 minutes and the bottom aqueous layer was removed. The aqueous layer was extracted with 2.0 L methyl tert-butyl ether, the methyl tert-butyl ether was combined and washed with brine solution and water. The methyl tert-butyl ether was distilled out and 8.0 L hot water was added to remove traces of the methyl tert-butyl ether. The slurry was cooled down to 20-25° C., filtered and washed with 1 L water. The wet product was dried at 50-55° C. until the moisture content was less than 2%. Dry weight=0.95 to 0.96 kg.

### Example 7

### Preparation of Clarithromycin

1.0 Kg SMOP oxime was suspended in 3.0 L ethyl alcohol. 3.0 L Water was added at 25-35° C. Then 1.165 kg sodium metabisulphite was added and 0.159 kg formic acid was added to adjust the pH to 3.8-4.1. The temperature of the mass was raised to 60° C. The mass was stirred at 57-63° C. for 5.0 hours, and then cooled to a temperature of 50-55° C. 1.165 Kg Sodium metabisulphite was added. The mass was heated to 60° C. The mass was stirred and the temperature of the reaction was maintained at 57-63° C. for 5.0 hours. The mass was cooled to 30-35° C. and a 50% NaOH solution was slowly added to adjust the pH to 10.5-11.0. The mass was stirred at 30-35° C. for 30 min. The slurry was filtered and washed with 0.25 L ethanol+water (0.50+0.60). The wet solid was stirred with 8.0 L water at 30-40° C. The product was dried until the moisture content was less than 2%. Dry weight=0.57 to 0.60 kg.

### Example 8

### Purification of Clarithromycin

14 L Ethanol and 1.0 kg crude clarithromycin were charged in a reactor. The mass was heated to reflux. 0.1 Kg Carbon and 0.1 kg hyflow were added. The carbon cake was filtered and washed with 1.0 L hot ethanol. The filtrate was collected and cooled up to 0-5° C. The filtrate was then stirred at 0-5° C. for 2 hrs. The product was filtered and dried at 70-75° C. until the LOD was less than 2%. Dry weight=0.87 kg.

1. A process involving a one-pot reaction for preparing erythromycin 9-oxime salt comprising:
(a) reacting erythromycin thiocyanate with an ammonium source to obtain erythromycin free base; and
(b) oximating the C-9 carbonyl of the erythromycin free base by reacting the erythromycin free base with triethylamine and hydroxyl amine hydrochloride to form the erythromycin oxime salt.

2. The process of claim 1, wherein the reaction in step (a) is conducted in at least one organic solvent.

3. The process of claim 2, wherein the at least one organic solvent dichloromethane.

4. The process of claim 1, wherein in step (a) an organic layer is removed from an aqueous layer and further distilled under reduced pressure.

5. The process of claim 1, wherein the reaction of step (b) is conducted in at least one organic solvent.

6. The process of claim 5, wherein the at least one organic solvent is C₁-C₄ alcohols.

7. The process of claim 6, wherein the at least one organic solvent is methanol.

8. The process of claim 1, wherein the reaction mixture in step (b) is heated to about reflux for about 20 hours to about 24 hours.

9. The process of claim 8, wherein the heated reaction mixture is washed with methanol at a temperature of about 15° C. or less.

10. The process of claim 1, wherein the ammonium source is an about 25% (v/v) liquid ammonia solution.

11. The process of claim 1, wherein the erythromycin 9-oxime salt is erythromycin oxime hydrochloride.

12. A process involving a one-pot reaction preparing clarithromycin, comprising the process of claim 1 further comprising, after step (b):
(c) reacting the erythromycin oxime obtained in step (b) with an ammonium source to obtain erythromycin 9-oxime salt;
(d) silylating the hydroxy groups at the oxime group, and the 2′ and 4″ positions of the erythromycin 9-oxime salt to obtain a silylated derivative;
(e) methylating the hydroxy group at the 6 position of the silylated derivative using at least one methylating agent in the presence of at least one inorganic base to obtain SMOP, wherein SMOP is 6-O-methyl-2′,4″-bis(trimethylsilyl)-erythromycin A 9-O-(2-methoxyprop-2-yl)oxime; and
(f) converting the SMOP into clarithromycin using at least one deoximating agent in the presence of aqueous ethanol.

13. The process of claim 12, wherein the reaction in step (c) is conducted in at least one organic solvent.

14. The process of claim 12, wherein the at least one organic solvent is dichloromethane.

15. The process of claim 12, wherein the ammonium source in step (c) is an about 25% (v/v) liquid ammonia solution.

16. The process of claim 12, wherein the ammonia solution is added in a dropwise manner in step (c).

17. The process of claim 12, wherein step (d) is conducted by silylating the hydroxy groups using hexamethyl disilazene in the presence of methoxy propene and pyridine hydrobromide

18. The process of claim 12, wherein the reaction mixture in step (d) is maintained at a temperature of about 10° C. to about 20° C.

19. The process of claim 12, wherein the at least one organic solvent in step (e) comprises methyl tert-butyl ether.

20. The process of claim 12, wherein the at least one organic solvent in step (e) comprises methyl tert-butyl ether and another aprotic solvent.

21. The process of claim 20, wherein the another aprotic solvent is dimethylsulfoxide.

22. The process of claim 12, wherein the at least one inorganic base in step (e) is a base selected from a group consisting of potassium hydroxide, sodium hydroxide, potassium hydride, sodium hydride, potassium tert-butoxide and sodium tert-butoxide.

23. The process of claim 12, wherein the at least one inorganic base is potassium hydroxide.

24. The process of claim 12, wherein the at least one methylating agent in step (e) is selected from the group consisting of methyl iodide, methyl bromide, dimethylsulfate, methyl p-toluenesulfonate, methyl methanesulfonate and dimethyl sulfate.

25. The process of claim 24, wherein the methylating agent is methyl iodide.

26. The process of claim 12, wherein the reaction mixture in step (e) is maintained at a temperature of about 10° C. to about 20° C.

27. The process of claim 12, wherein the at least one deoximating agent in step (f) comprises an inorganic sulfur oxide compound.

28. The process of claim 27, wherein the inorganic sulfur oxide compound is selected from the group consisting of sodium hydrogen sulfite, sodium pyrosulfate, sodium thiosulfate, sodium sulfite, sodium hydrosulfite, sodium metabisulfite, sodium dithionate, potassium hydrogen sulfite, potassium thiosulfate and potassium metabisulfite.

29. The process of claim 28, wherein the at least one deoximating agent is sodium metabisulphite.

30. The process of claim 12, wherein the amount of the at least one deoximating agent in step (f) is about 1 to about 10 molar equivalents, relative to the SMOP.

31. The process of claim 30, wherein the amount of the at least one deoximating agent in step (f) is about 4 to about 7 molar equivalents, relative to the SMOP.

32. The process of claim 12, wherein step (f) is conducted by carrying out at least the following steps:
(f)(1) reacting the SMOP with the at least one deoximating agent and at least one acid in the presence of aqueous ethanol at an ethanol/water ratio of about 1:1 (v/v);
(f)(2) heating the mixture from step (f)(1) to about 50° C. to about 65° C.;
(f)(3) cooling the heated reaction mixture from step (f)(2); and
(f)(4) adding sodium hydroxide to obtain the clarithromycin.

33. The process of claim 32, wherein the at least one acid in step (f)(1) is formic acid.

34. The process of claim 32, wherein the at least one acid is added in step (f)(1) until the pH of the reaction mixture reaches about 3.5 to about 4.5.

35. The process of claim 32, wherein the heated reaction mixture is cooled to a temperature ranging from about 30° C. to about 35° C. in step (f)(3).

36. The process of claim 32, wherein in step (f)(4) sodium hydroxide is added until the pH of the reaction mixture reaches about 10 to about 11.

37. The process of claim 32, wherein in step (f)(4) sodium hydroxide is added until the pH of the reaction mixture reaches about 10.2 to about 10.5.

38. The process of claim 12, wherein the yield is above 54%.
